(19) United States
(12) Patent Application Publication
(10) Pub. No.: US 2022/0273736 A1
(43) Pub. Date: Sep. 1, 2022

(54) METHODS OF DETECTING OVARIAN CANCER

(71) Applicant: UCL BUSINESS LTD, London (GB)

(72) Inventors: Martin Widschwendter, London (GB); Nuno Nene, London (GB)

(21) Appl. No.: 17/594,631

(22) PCT Filed: Apr. 23, 2020
(86) PCT No.: PCT/EP2020/061394

§ 371 Date: Oct. 25, 2021

(30) Foreign Application Priority Data
Apr. 26, 2019 (GB) 1905861.9

(51) Int. Cl. A61K 35/747; C12Q 1/6886; A61K 45/06; C12Q 1/689; A61K 9/00; G16B 30/10; G16H 10/40; G16H 50/20

(52) U.S. Cl. CPC A61K 35/747; C12Q 1/6886; A61K 45/06; C12Q 1/689; A61K 9/0034; G16B 30/10; G16H 10/40; G16H 50/20

(57) ABSTRACT

The invention relates to a method of determining whether a subject is at an increased risk of developing or having ovarian cancer or being a carrier of a germline BRCA1 and/or BRCA2 mutation. The method may comprise determining whether the percentage contribution of lactic acid-producing bacteria to the bacterial load of a sample obtained from the lower genital tract of the subject is more or less than a percentage threshold level. The subject is at increased risk if the percentage contribution of lactic acid-producing bacteria to the bacterial load of the sample is less than the percentage threshold level. Alternatively, the method may comprise determining the pH of a sample obtained from the lower genital tract of the subject. The subject is at increased risk if the pH is higher than a pH threshold level.

### INTRODUCTION

Ovarian cancer is the 8^{th} most common cancer among women worldwide, accounting for around 4% of women's cancers. As with all cancers, early diagnosis of ovarian cancer dramatically improves survival rates. Around 90% of women diagnosed with the earliest stage (stage 1) ovarian cancer survive their disease for at least 5 years compared to 10% for women diagnosed with the most advanced stage (stage 4) of disease.

Unfortunately, early symptoms of ovarian cancer are very vague, for example bloating, abdominal pain, and loss of appetite. Consequently, early diagnosis is extremely difficult and almost 75% of ovarian cancer cases are only diagnosed at a late stage (stage 3 or 4). Identification of women at high risk for having very early stage ovarian cancer or for developing ovarian cancer in the future would mean that such women could be screened earlier and monitored more frequently, thus leading to earlier diagnosis.

There are several factors which are known to increase the risk of developing ovarian cancer. In particular, women carrying a mutation in the BRCA1 or BRCA2 gene have a 34- and 13-fold increased risk of developing ovarian cancer by the age of 80, respectively. In addition, a family history of ovarian cancer, endometriosis, low parity, a previous breast cancer diagnosis, obesity, and combined (i.e. oestrogen and progesterone) hormone replacement therapy (HRT) are thought to contribute to the risk of developing ovarian cancer. Unfortunately, not many of these risk factors are modifiable. Some possible protective factors have also been identified, for example use of the combined oral contraceptive pill (OCP), having children, and fallopian tube ligation.

Nevertheless, the ability to identify women at high risk of ovarian cancer is still inadequate. Even in BRCA1 mutation carriers, 44% of whom develop ovarian cancer, it is difficult to predict which women will actually go on to develop the disease. Hence, there is still a great need for identification of additional risk factors and development of further predictive tools, as well as new strategies for preventing ovarian cancer.

### SUMMARY OF THE INVENTION

The inventors of the present invention have surprisingly found that the composition of a woman's lower genital tract microbiota and the pH of their lower genital tract are significantly associated with their risk of ovarian cancer and of carrying a germline BRCA1/2 mutation. For example, the inventors found that if less than 50% of a subject's cervico-vaginal microbiota is contributed by L. iners, L. crispatus, L. gasseri, and L. jensenii, then the subject is at an increased risk of having ovarian cancer or developing ovarian cancer in the future. In addition, it was found that if less than 50% of a subject's cervico-vaginal microbiota is contributed by L. iners, L. crispatus, L. gasseri, and L. jensenii, then the subject is at an increased risk of being a carrier of a germline BRCA1 or BRCA2 mutation.

The inventors have also determined that subjects having an elevated cervico-vaginal pH have an increased risk of having ovarian cancer or developing ovarian cancer in the future. The inventors have also determined that subjects having an elevated cervico-vaginal pH have an increased risk being a carrier of a germline BRCA1/2 mutation. This is thought to be because L. iners, L. crispatus, L. gasseri, and L. jensenii are lactic acid-producing bacteria, which play a major role in creating the acidic environment of the lower genital tract.

These new risk factors for ovarian cancer represent a much needed addition to the tools currently available for predicting women's risk of ovarian cancer. The ability to identifying women as being at an increased risk of ovarian cancer at an earlier stage and with improved accuracy will surely lead to earlier diagnosis, treatment, and survival. Advantageously and in contrast to most known risk factors, the risk factors identified by the inventors of the present invention are modifiable, since therapeutic intervention can alter a subject's microbiota.

In recent years, there has been a surge of research into the human microbiota and its significance in human health and it is now thought that gastrointestinal microorganisms could contribute to numerous diseases, including an estimated 20% of human malignancies. The human microbiota comprises the bacteria, archaea, fungi, protists, and viruses colonising the human body, including the skin, oral cavity, digestive tract, and urogenital system. However, since 99% of the body's microbial mass is found in the gut, research has been largely focussed on the gut microbiota and its role in diseases such as colon cancer.

In comparison to the gut, little is known about the microbiota of the lower female genital tract and its significance in human health. It is known that the vagina is often colonised by Lactobacillus species, which contribute to the low pH of the vagina through production of lactic acid. To better understand the normal composition of the microbiota in the lower genital tract of healthy women, Ravel et al. (2011) characterised the vaginal microbiome of asymptomatic, reproductive-age women using 16S rRNA sequencing. They found that the bacterial communities clustered into five groups; four groups were dominated by L. iners, L. crispatus, L. gasseri, or L. jensenii, respectively, whereas the other group had lower proportions of lactic acid-producing bacteria and higher proportions of anaerobic organisms. More recently, it has been suggested that the composition of the microbiota of the lower female genital tract may be associated with cervical cancer. However, there have been contradictory reports regarding which species of bacteria may be risk factors and which may be protective factors for cervical cancer and a consensus has not yet been reached.

Whilst it is well known that the lower female genital tract (including the cervix, endocervix, cervico-vagina, vagina, and/or vulva) is colonised with bacteria in healthy women, there are few reports of bacteria being detected in the upper female genital tract (including the uterus, fallopian tubes, fimbriae, and/or ovaries) and it is thought that such bacteria are occasional, opportunistic organisms rather than commensals. In view of the distance between the lower female genital tract and the ovaries and fallopian tubes (from which most ovarian cancers originate), and the clear distinction between the type and quantity of bacteria in these regions, the inventors were highly surprised to find that the microbiota of the lower female genital tract was dysregulated in women having ovarian cancer. Even more surprisingly, the inventors found that the microbiota of the lower female genital tract was dysregulated in carriers of germline BRCA1 mutations, who have not yet developed ovarian cancer but are at a very high risk of developing ovarian cancer in the future.

The fact that the new risk factors for ovarian cancer described in this application can be assessed using samples obtained in a minimally invasive manner (for example swabbing of the lower female genital tract, which can even be done by the subject themselves), is a further advantage because it means that uptake of these predictive tests by subjects is likely to be very high.

Moreover, the fact these risk factors are modifiable—unlike many of the known risk factors—means that subjects identified as being at an increased risk of developing ovarian cancer can be treated to decrease their risk of developing the disease in the future. Such a treatment is hereinafter referred to as a prophylactic treatment.

### STATEMENTS OF INVENTION

The present invention provides a method of determining whether a subject is at an increased risk of developing or having ovarian cancer or being a carrier of a germline BRCA1 and/or BRCA2 mutation, the method comprising:
determining whether the percentage contribution of lactic acid-producing bacteria to the bacterial load of a sample obtained from the lower genital tract of the subject is more or less than a percentage threshold level,
wherein the subject is at increased risk of developing or having ovarian cancer or being a carrier of a germline BRCA1 and/or BRCA2 mutation if the percentage contribution of lactic acid-producing bacteria to the bacterial load of the sample is less than the percentage threshold level,
and wherein the subject is human and female.

The present invention also provides a method of identifying a subject expected to benefit from modification of their lower genital tract microbiota, the method comprising:
determining whether the percentage contribution of lactic acid-producing bacteria to the bacterial load of a sample obtained from the lower genital tract of the subject is more or less than a percentage threshold level,
wherein the subject is expected to benefit from modification of their lower genital tract microbiota if the percentage contribution of lactic acid-producing bacteria to the bacterial load of the sample is less than the percentage threshold level,
and wherein the subject is human and female.

The lactic acid-producing bacteria can be:
b) lactic acid-producing Lactobacillus species, or
b) one or more species selected from the group consisting of L. crispatus, L. iners, L. gasseri, and L. jensenii.

The percentage threshold level can be:
a) a value from 30 to 70%,
b) a value from 40 to 60%,
c) a value from 45 to 55%,
d) a value from 48 to 52%,
e) 50%, or
f) the minimum percentage contribution of lactic acid-producing bacteria to the bacterial load of lower genital tract samples in a healthy control population.

The percentage contribution of lactic acid-producing bacteria to the bacterial load of the sample can be determined using:
a) targeted amplicon sequencing, optionally 16S rRNA amplicon sequencing, or
b) metagenomic sequencing, optionally shotgun metagenomic sequencing.

The present invention further provides a method of determining whether a subject is at an increased risk of developing or having ovarian cancer or being a carrier of a germline BRCA1 and/or BRCA2 mutation, the method comprising:
determining the pH of a sample obtained from the lower genital tract of the subject,
wherein the subject is at increased risk of developing or having ovarian cancer or being a carrier of a germline BRCA1 and/or BRCA2 mutation if the pH is higher than a pH threshold level,
and wherein the subject is human and female.

The present invention also provides a method of identifying a subject expected to benefit from modification of their lower genital tract microbiota, the method comprising:
determining the pH of a sample obtained from the lower genital tract of the subject,
wherein the subject is expected to benefit from modification of their lower genital tract microbiota if the pH is higher than a pH threshold level,
and wherein the subject is human and female.

The pH threshold level can be:
a) a pH from 4.0 to 6.0,
b) a pH from 4.5 to 5.5,
c) a pH from 5.0 to 5.5, or
d) a value 0.3-0.6 pH units higher than the average pH of samples obtained from a healthy control population.

Wherein the method is a method of determining whether a subject is at an increased risk of developing or having ovarian cancer, the subject can have previously been identified as a carrier of a germline BRCA1 and/or BRCA2 mutation.

The subject can be younger than 60, 55, 50, 45, or 40 years old.

The subject can be younger than 55 years old.

The subject can be younger than 40 years old.

The subject can be identified as being at a further increased risk of developing or having ovarian cancer or being a carrier of a germline BRCA1 and/or BRCA2 mutation if:
a) the subject has two or more first degree family members having a history of ovarian or breast cancer;
b) the subject has never used oral contraceptive pills (OCPs) or has used OCPs for a total of less than 5 years; and/or
c) the subject has used combined hormone replacement therapy (HRT) or has used combined HRT for a total of more than 5 years.

The modification can be an increase in the contribution of lactic acid-producing bacteria to the bacterial load of their lower genital tract microbiota, optionally the lactic acid-producing bacteria can be:
a) lactic acid-producing Lactobacillus species, or
b) one or more species selected from the group consisting of L. crispatus, L. iners, L. gasseri, and L. jensenii.

The modification can be an increase in the contribution of lactic acid-producing bacteria to the bacterial load of their lower genital tract microbiota such that the total percentage contribution of the lactic acid-producing bacteria to the bacterial load of their lower genital tract microbiota is:
a) at least 30%,
b) at least 40%,
c) at least 45%,
d) at least 48%,
e) at least 50%, or
f) at least the minimum percentage contribution of lactic acid-producing bacteria to the bacterial load of lower genital tract samples in a healthy control population.

The benefit can be a decrease in the risk of the subject developing ovarian cancer.

The present invention further provides a composition for use in a method of decreasing the risk of a subject developing ovarian cancer, wherein the subject has been identified as being at increased risk of developing ovarian cancer or as being expected to benefit from modification of their lower genital tract microbiota using any of the methods described herein, wherein the composition comprises:
a) one or more species of lactic acid-producing bacteria,
b) an oestrogen, or
c) an oral contraceptive,
or any combination thereof.

The composition can comprise one or more species of lactic acid-producing bacteria selected from the group consisting of:
a) lactic acid-producing Lactobacillus species, or
b) L. crispatus, L. iners, L. gasseri, and L. jensenii.

The method can comprise administration of the composition to the lower genital tract.

The present invention further provides a vaginal suppository comprising any of the compositions described herein for any of the uses described herein.

Optionally, the present invention provides a method of determining whether a subject is at an increased risk of developing or having ovarian cancer or being a carrier of a germline BRCA1 and/or BRCA2 mutation, the method comprising:
determining whether the percentage contribution of L. crispatus, L. iners, L. gasseri, and L. jensenii to the bacterial load of a sample obtained from the lower genital tract of the subject is more or less than 50%,
wherein the subject is at increased risk of developing or having ovarian cancer or being a carrier of a germline BRCA1 and/or BRCA2 mutation if the percentage contribution of L. crispatus, L. iners, L. gasseri, and L. jensenii to the bacterial load of the sample is less than 50%,
and wherein the subject is human and female and is 55 years old or younger.

Optionally, the present invention provides a method of determining whether a subject is at an increased risk of developing or having ovarian cancer or being a carrier of a germline BRCA1 and/or BRCA2 mutation, the method comprising:
determining the pH of a sample obtained from the lower genital tract of the subject,
wherein the subject is at increased risk of developing or having ovarian cancer or being a carrier of a germline BRCA1 and/or BRCA2 mutation if the pH is higher than 5.0,
and wherein the subject is human and female and is 55 years old or younger.

The present invention further provides a method for decreasing the risk of a subject developing ovarian cancer, the method comprising administering an effective amount of one or more lactic acid-producing bacteria to the subject,
wherein the subject is a female human and has been identified as being at an increased risk of developing ovarian cancer by:
determining whether the percentage contribution of lactic acid-producing bacteria to the bacterial load of a sample obtained from the lower genital tract of the subject is more or less than a percentage threshold level, and
identifying the subject as being at an increased risk of developing ovarian cancer if the percentage contribution of lactic acid-producing bacteria to the bacterial load of the sample is less than the percentage threshold level.

The present invention further provides a method for decreasing the risk of a subject developing ovarian cancer, whereinin the subject is at increased risk for developing ovarian cancer, the method comprising:
a) obtaining or receiving a sample from the lower genital tract of a subject,
b) determining whether the percentage contribution of lactic acid-producing bacteria to the bacterial load of a sample obtained from the lower genital tract of the subject is more or less than a percentage threshold level,
c) identifying the subject as being at an increased risk of developing ovarian cancer if the percentage contribution of lactic acid-producing bacteria to the bacterial load of the sample is less than the percentage threshold level,
d) administering an effective amount of one or more lactic acid-producing bacteria to the subject identified as being at an increased risk of ovarian cancer,
wherein the subject is human and female.

The present invention further provides a method for decreasing the risk of a subject developing ovarian cancer, the method comprising administering an effective amount of one or more lactic acid-producing bacteria to the subject,
wherein the subject is a female human and has been identified as being at an increased risk of developing ovarian cancer by:
determining the pH of a sample obtained from the lower genital tract of subject, and identifying the subject as being at an increased risk of developing ovarian cancer if the pH is higher than a pH threshold level.

The present invention further provides a method for decreasing the risk of a subject developing ovarian cancer, whereinin the subject is at increased risk for developing ovarian cancer, the method comprising:
a) obtaining or receiving a sample from the lower genital tract of a subject,
b) determining the pH of a sample obtained from the lower genital tract of the subject,
c) identifying the subject as being at an increased risk of developing ovarian cancer if the pH is higher than a pH threshold level,
d) administering an effective amount of one or more lactic acid-producing bacteria to the subject identified as being at an increased risk of ovarian cancer,
wherein the subject is human and female.

The present invention further provides a computer program comprising instructions which, when the program is executed by a computer, cause the computer to carry out any of the methods described herein.

The present invention also provides a computer program comprising instructions which, when the program is executed by a computer, cause the computer to carry out the following steps:
processing bacterial nucleic acid sequences derived from a sample obtained from the lower genital tract of a subject to determine whether the percentage contribution of lactic acid-producing bacteria to the bacterial load of the sample is more or less than a percentage threshold level, and
identifying the subject as being at increased risk of developing or having ovarian cancer or being a carrier of a germline BRCA1 and/or BRCA2 mutation if the percentage contribution of lactic acid-producing bacteria to the bacterial load of the sample is less than the percentage threshold level,
wherein the subject is human and female.

### BRIEF DESCRIPTION OF FIGURES

FIG. 1. Sum of proportions of community-type L species with Age, two-dimensional fitted density of individuals. A. Controls in the OC set. B. Controls in the BRCA1 set. Colour bars represent density and are the same for all panels in this figure. The higher the density the closer the colour is to blue. Sum of proportions, y-axis. Age, x-axis. Densities were estimated with the kde2d package in R.

FIG. 2. Overall species abundance per subject for the Ovarian Cancer (OC) and cancer-free Control set. Heatmap colours are proportional to abundance in each subject. The legend represents membership of community-types L or O and if the subject is a Case or a Control. <50 and >50 indicates the age of the individuals, younger or older than 50 years. SDI, Shannon Diversity Index.

FIG. 3. Forest Plots including Odds Ratio (OR), 95% Confidence Interval (95% CI) and p-values for selected covariates used in the Ovarian Cancer (OC)/cancer-free Control set. A. and B. Age <50 years. Community-type O association is also shown adjusted for “Duration of OCP use” and “Duration of combined hormone use (years)”. C and D. Age >50 years. For a truncated confidence interval, an arrow ending is used. BMI, Body Mass Index. OCP, Oral Contraceptive Pill.

FIG. 4. Overall species abundance per subject for the BRCA1 mutation carriers and BRCA1 wildtype Control set. Heatmap colours are proportional to abundance in each subject. The legend represents membership of community-types L or O and if the subject is a Case or a Control. Within the mutation carrier cases, those individuals who have more than one first degree relative affected with cancer are labelled mut>1. Otherwise they are labelled as mut. <50 and >50 indicates the age of the individuals, younger or older than 50 years. SDI, Shannon Diversity Index.

FIG. 5. Forest Plots including Odds Ratio (OR), 95% Confidence Interval (95% CI) and p-values for selected covariates used in the BRCA1 mutation carrier and BRCA1 wild type control set.

A. and B. Age <50 years. C and D. Age >50 years. For a truncated confidence interval, an arrow ending is used.BMI, Body Mass Index. OCP, Oral Contraceptive Pill.

FIG. 6. Forest Plots including Odds Ratio (OR), 95% Confidence Interval (95% Cl) and p-values for prediction of community type for different age thresholds.

A. Ovarian Cancer data set B. BRCA set. For a truncated confidence interval, an arrow ending is used.

### DESCRIPTION

### The Microbiota of the Lower Female Genital Tract

The lower female genital tract is occupied by commensal bacteria, often including lactic acid-producing bacteria such as species of the genus Lactobacillus. It is known that the overgrowth of certain bacteria in the lower female genital tract can result in diseases such as bacterial vaginosis (BV).

The inventors of the present invention have surprisingly found that the composition of the microbiota of a subject's lower female genital tract is associated with the subject's risk of having or developing ovarian cancer, as well as their risk of being a BRCA1 and/or 2 germline mutation carrier. In particular, subjects whose lower genital tract microbiota comprised less than 50% lactic acid-producing bacteria were found to have an increased risk of having or developing ovarian cancer or of being a BRCA1/2 germline mutation carrier. The inventors consider that a microbiota dominated with lactic acid-producing bacteria is protective against ovarian cancer.

As used herein, “microbiota” refers to the community of bacteria occupying a particular environment.

As used herein, the “lower genital tract” or “lower female genital tract” of a subject includes the subject's cervix, endocervix, cervico-vagina, vagina, and/or vulva.

### The Subject

The present invention relates to the identification of subjects at an increased risk of having or developing ovarian cancer. Hence, in the present invention, the subject is a woman. The subject is human and female. The subject has tissue(s) capable of developing into ovarian cancer (for example, elium, fallopian tube tissue, Mullerian Duct remnants, and/or peritoneal lining of the pelvis). Optionally, the subject has one or more ovaries and/or one or more fallopian tubes.

The inventors of the present invention have shown that the there is a statistically significant association between the percentage contribution of lactic acid-producing bacteria to the bacterial load of a sample obtained from the lower female genital tract of a subject and the subject's risk of having or developing ovarian cancer or of being a germline BRCA1/2 mutation carrier, when the subject is younger than 55 years old. The inventors did not test ages above 55 years old due to an insufficient number of samples, but consider that a significant association would be observed in subjects younger than 60 years old. Hence, in the present invention, the subject may be younger than 60, 55, 50, 45, or 40 years old. Preferably, the subject is younger than 55 years old.

The inventors have also shown that the younger the subject, the more meaningful the test result. In particular, Example 4 demonstrates that the odds ratio (OR) is highest for subjects younger than 40 years old. Hence, even more preferably, the subject is younger than 40 years old. The subject may be pre-menopausal or post-menopausal.

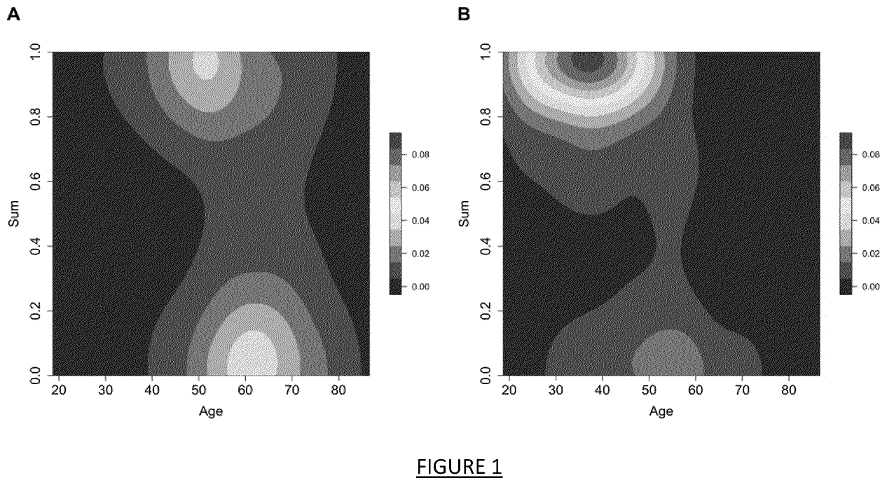

It has been found that control populations of subjects generally cluster into two groups, one having a higher percentage contribution of lactic acid-producing bacteria in their lower genital tract, the other having a lower percentage contribution of lactic acid-producing bacteria in their lower genital tract (see FIG. 1). Therefore, the subject may be younger than an age threshold which is able to distinguish between two such clusters. For example, the inventors of the present invention initially studied subjects younger than 50 years old, since this age threshold is able to distinguish between the clusters associated with lower and higher percentages of L. crispatus, L. iners, L. gasseri, and L. jensenii in a control population (see FIG. 1).

The subject may be post-menarcheal. The subject may be 16 years old or older, or 18 years old or older.

Conveniently, the subject had not been menstruating at the time the sample was obtained. The subject may not have been pregnant at the time the sample was obtained. Preferably, the subject was not taking antibiotics at the time the sample was obtained. More preferably, the subject had not taken antibiotics for at least 30 days before the sample was obtained. The subject may have had no diagnosed gynaecological condition at the time the sample was obtained. The subject may have had a benign gynaecological condition at the time the sample was obtained.

The subject may be Asian, Black, Hispanic, or Caucasian. The subject may be Caucasian.

### Sampling the Microbiota of the Subject's Lower Genital Tract

The microbiota of a sample obtained from a subject's lower female genital tract represents the microbiota of the subject's lower female genital tract. As used herein, “a sample obtained from the subject's lower genital tract” means a sample previously obtained from the subject's cervix, endocervix, cervico-vagina, vagina, vulva, or any combination thereof.

The sample may comprise secretions and/or discharge from the lower female genital tract. The sample may be a fluid. The sample may comprise bacterial cells, fungal cells, viruses, and/or human cells, for example epithelial cells and/or immune cells.

In aspects of the invention involving determining whether the percentage contribution of lactic acid-producing bacteria to the bacterial load of a sample obtained from the lower genital tract of the subject is more or less than a percentage threshold value, the sample comprises a bacterial load that is sufficient to perform the determination.

In aspects of the invention involving determining the pH of a sample obtained from the lower genital tract of the subject, the sample allows for determination of the pH of the sample. The sample is preferably a sample of vaginal fluid.

Samples may be obtained or may have been obtained using methods known to the person skilled in the art. Samples may be obtained or may have been obtained by washing i.e. rinsing the lower genital tract with a washing buffer which is subsequently collected. Samples may be obtained or may have been obtained by wicking, for example inserting tampons, strips, sponges, or other suitable materials into the lower genital tract to absorb fluids. Samples may be obtained or may have been obtained using diaphragm-like devices which can be placed over the cervix to collect a sample. Preferably, samples may be obtained or may have been obtained by swabbing, for example using a swab, brush, broom, spatula, or other suitable device. Samples may be obtained or may have been obtained using the ThinPrep™ or ESwab™ systems.

Advantageously, samples may be obtained or may have been obtained by the subject themselves i.e. self-sampling. The inventors consider that self-sampling results in improved uptake of the test since samples need not be taken by a medical professional or in a clinic, but rather can be taken at home and sent to the testing facility by post.

### Percentage Contribution of Lactic Acid-Producing Bacteria

The inventors of the present invention found that the risk of having or developing ovarian cancer or of being a germline BRCA1/2 mutation carrier is associated with the percentage contribution of lactic acid-producing bacteria to the lower female genital tract microbiota.

### Lactic Acid-Producing Bacteria

As used herein, “lactic acid-producing bacteria” may mean bacteria which are capable of producing lactic acid. Preferably, “lactic acid-producing bacteria” means bacteria which characteristically produce lactic acid when inhabiting the lower female genital tract.

The lactic acid-producing bacteria may be of the order Lactobacillales. Preferably, the lactic acid-producing bacteria are of the genus Lactobacillus. Most preferably, the lactic acid-producing bacteria are L. crispatus, L. iners, L. gasseri, and/or L. jensenii. The lactic acid-producing bacteria may include L. acidophilus, L. amylovorus, L. gallinarum, L. johnsonii, and/or L. vaginalis.

As used herein, the term “community-type L” (i.e. “community-type lactobacilli”) refers to a microbiota dominated by (i.e. comprising ≥50%) L. crispatus, L. iners, L. gasseri and/or L. jensenii. The microbiota may be dominated by one, two, three, or four species selected from the group consisting of L. crispatus, L. iners, L. gasseri and L. jensenii.

As used herein, the term “community-type O” (i.e. “community-type other”) refers to a microbiota dominated by (i.e. comprising ≥50%) one or more species other than L. crispatus, L. iners, L. gasseri and L. jensenii. In other words, “community-type O” refers to a community wherein <50% of the bacteria are contributed by L. crispatus, L. iners, L. gasseri and/or L. jensenii.

### Determining the Percentage Contribution of Lactic Acid-Producing Bacteria

The present invention involves determining the percentage contribution of one or more bacterial species to the bacterial load of a sample obtained from the lower female genital tract of the subject.

As used herein, “bacterial load” means the measurable quantity of bacteria in an environment. As used herein, the “percentage contribution of lactic acid producing bacteria to the bacterial load of a sample” means the total (i.e. sum, cumulative) percentage of the total bacterial load of the sample which is lactic acid-producing bacterial species.

The person skilled in the art understands that the percentage contribution of lactic acid- producing bacteria to the bacterial load of a sample may be entirely contributed by one species of lactic acid-producing bacteria, or may be contributed by two, three, four, or more species of lactic acid-producing bacteria.

The person skilled in the art understands that, in order to determine whether the percentage contribution of lactic acid-producing bacteria to the bacterial load of a sample is more or less than a percentage threshold, it is not always necessary to determine the percentage contribution of each and every bacterial species in the sample. If, for example, it is determined that the percentage contribution of a single lactic acid-producing bacterial species to the bacterial load of a sample is more than the percentage threshold, no further analysis need be done. Similarly, if it is determined that the percentage contribution of one non-lactic acid-producing bacterial species to the bacterial load of a sample is more than the percentage threshold, no further analysis need be done.

The invention may involve determining the percentage contribution of one, two, three, four, or more lactic acid-producing bacterial species (preferably Lactobacillus species, more preferably species selected from the group consisting of L. crispatus, L. iners, L. gasseri and L. jensenii) to the bacterial load of a sample obtained from the lower genital tract of the subject.

The percentage contribution of lactic acid-producing bacteria to the bacterial load of a sample may be determined by methods known to the person skilled in the art. Optionally, the percentage contribution of lactic acid-producing bacteria to the bacterial load of a sample may be determined by visual inspection by microscopy, metagenomics sequencing (for example shotgun metagenomics sequencing), or targeted amplicon sequencing (for example 16S rRNA gene sequencing). Preferably, the percentage contribution of lactic acid-producing bacteria to the bacterial load of a sample is determined by 16S rRNA gene sequencing, most preferably by sequencing of the hyper-variable V1-V3 regions of bacterial SSU rDNA (16S rRNA genes).

Next generation sequencing may be used. For example, Illumina™ sequencing may be used. Taxonomic classification may be based on NCBI's The Taxonomy Database.

### Identifying Whether a Subject is at Increased Risk

The present invention further involves determining whether the percentage contribution of lactic acid-producing bacteria to the bacterial load of a sample obtained from the lower genital tract of the subject is more or less than a percentage threshold level.

The subject is identified as being at an increased risk of developing or having ovarian cancer or being a carrier of a germline BRCA1 and/or BRCA2 mutation if the percentage contribution of lactic acid-producing bacteria to the bacterial load of the sample is less than a percentage threshold level. The subject may be identified as not being at an increased risk of developing or having ovarian cancer or being a carrier of a germline BRCA1 and/or BRCA2 mutation if the percentage contribution of lactic acid-producing bacteria to the bacterial load of the sample is equal to or more than a percentage threshold level.

As used herein, “increased risk” may mean increased risk as compared to the average risk of subjects in a control population, preferably a matched control population. As used herein, “increased risk” may mean a higher risk than the average human female. The risk of a human female developing ovarian cancer in their lifetime is about 1.5%. Therefore, “increased risk” may mean a lifetime risk of above about 1.5%, above about 2.0%, above about 2.5%, or above about 3.0%. Preferably, “increased risk” may mean a lifetime risk of above about 1.5%. “Increased risk” may be mean a statistically significantly increased risk.

### The Percentage Threshold Level

The inventors of the present invention have shown that if the (sum) percentage contribution of L. crispatus, L. iners, L. gasseri and L. jensenii to the bacterial load of a sample obtained from the lower genital tract of the subject is less than a percentage threshold level of 50%, the subject is at an increased risk of having or developing ovarian cancer or of being a germline BRCA1/2 mutation carrier. Hence, if the (sum) percentage contribution of one or more of L. crispatus, L. iners, L. gasseri or L. jensenii to the bacterial load of a sample obtained from the lower genital tract of the subject is equal to or more than a percentage threshold level of 50%, the subject is not at an increased risk of having or developing ovarian cancer or of being a germline BRCA1/2 mutation carrier.

The inventors found that control populations of subjects generally cluster into two groups, one having a higher percentage contribution of lactic acid-producing bacteria in their lower genital tract, the other having a lower percentage contribution of lactic acid-producing bacteria in their lower genital tract. Hence, the inventors have determined that any percentage threshold level that is able to distinguish between two such clusters may be used. The percentage threshold may be 30, 35, 40, 45, 50, 55, 60, 65, or 70%. Preferably, the percentage threshold level is a value from 30 to 70%, more preferably from 40 to 60%, even more preferably from 45 to 55%, and even more preferably from 58 to 42%. Advantageously, the percentage thresholds is about 50%.

The inventors found that community-type O cervico-vagina microbiota dominates in older women and that the detection of this community-type in younger women is a risk factor for having or developing ovarian cancer or for being a BRCA1/2 germline mutation carrier. The inventors consider that such premature aging of the lower genital tract microbiota is causally involved in the genesis of ovarian cancer. Hence, in the present invention, the subject may be identified as being at an increased risk of having or developing ovarian cancer or for being a BRCA1/2 germline mutation carrier if they have a prematurely aged microbiota.

### The pH of the Lower Female Genital Tract

The lower female genital tract is an acidic environment, usually having a pH between approximately 4.0 and 5.0 in healthy women. This low pH is primarily due to the presence of lactic acid, and the main source of this lactic acid is lactic acid-producing bacteria such as Lactobacillus species. Ravel et al. (2011) observed that vaginal communities dominated by the common Lactobacillus species L. crispatus, L. iners, L. gasseri, or L. jensenii (groups I, III, II, and V, respectively, in Ravel) had a significantly lower pH than communities dominated by other species (group IV in Ravel). Hence, the present inventors determined that pH can be used as a surrogate measure of the percentage contribution of lactic acid-producing bacteria to the bacterial load of the lower genital tract.

### The pH Threshold Level

In the present invention, the subject is identified as being at increased risk of developing or having ovarian cancer or being a carrier of a germline BRCA1 and/or BRCA2 mutation if the pH of the sample is higher than a pH threshold level.

The pH threshold level may be a value from 4.0 to 6.0, preferably from 4.5 to 5.5, more preferably from 5.0 to 5.5. The pH threshold may be 4.5, 4.6, 4.7, 4.8, 4.9, 5.0, 5.1, 5.2, 5.3, 5.4, or 5.5.

The pH threshold level may be 0.3-0.6 pH units higher than the average pH of samples taken from a control population, optionally 0.3, 0.4, 0.5, or 0.6 pH units higher than the average pH of samples taken from a control population. Advantageously, the control population is matched by age and/or ethnicity. Preferably, the control population is healthy i.e. the subjects in the population do not have a gynaecological condition at the time the sample was obtained.

### Determining pH

The pH of the lower genital tract may be determined using methods known to a person skilled in the art. The pH of the lower genital tract may be determined using pH paper or dipsticks.

### In Vitro Methods

The determination of risk of the present invention is carried out entirely in vitro. In particular, the samples may have already (previously) been obtained from the human body.

### Controls

As used herein, “increased risk” may mean increased risk as compared to the average risk of subjects in a control population, preferably a matched control population.

A control population may be a healthy population i.e. a population of subjects not having ovarian cancer. A control population may be a population of subjects who have previously been identified as not being at an increased risk of having or developing ovarian cancer or of being a BRCA1/2 germline mutation carrier, optionally by belonging to a cluster of subjects wherein community-type L dominates their lower genital tract microbiota.

A control population may be matched to the subject based on other ovarian cancer risk-factors and/or protective factors (for example BRCA1/2 mutation status, age, number of first degree family members having a history of breast and/or ovarian cancer, previous breast cancer diagnosis, obesity, endometriosis, parity, OCP use, fallopian tube ligation, and/or HRT use). A control population may be matched to the subject based on age, ethnicity, country of residence, and/or menopausal status. Preferably, a control population is matched to the subject based on age and/or ethnicity.

### Further Risk Factors

In the present invention, the subject may have one or more other risk factors for having or developing ovarian cancer or for being a germline BRCA1/2 mutation carrier and hence may be at a further increased risk. For example;
the subject may have two or more first degree family members having a history of ovarian or breast cancer;
the subject may have never used oral contraceptive pills (OCPs) or may have used OCPs for a total of less than 5 years;
the subject may have used combined (i.e. containing both oestrogen and progesterone) hormone replacement therapy (HRT) or may have used combined HRT for a total of more than 5 years;
the subject may have had a previous breast cancer diagnosis;
the subject may be obese;
and/or
   the subject may have/have had endometriosis.

### BRCA1 and BRCA2

The inventors of the present invention have shown that the percentage contribution of lactic acid-producing bacteria to the bacterial load of a sample obtained from the lower genital tract of a subject is significantly associated with germline BRCA1 mutation status. They have also determined that the pH of sample obtained from the lower genital tract of a subject is significantly associated with germline BRCA1 mutation status.

The inventors consider that these findings are also applicable to germline BRCA2 mutation carriers because the mechanisms by which BRCA1 and BRCA2 mutations result in cancers are thought to be highly related. The BRCA1 and BRCA2 genes are both tumour suppressor genes and carriers of germline mutations in the BRCA1 and/or BRCA2 genes both have a dramatically increased risk of developing breast and ovarian cancer. Carriers of both of these mutations experience similar cell-non-autonomous changes. In particular, carriers of both BRCA1 and BRCA2 germline mutations experience higher titres of oestrogen and in particular progesterone at a certain phase during the menstrual cycle, which are risk factors for breast cancer (Widschwendter et al. (2013) The Lancet Oncology).

### Ovarian Cancer

As used herein, “ovarian cancer” may include fallopian tube cancers and primary peritoneal cancers and may include cancers which have developed from the ovarian surface epithelium, fallopian tube tissue, Mullerian Duct remnants, and/or peritoneal lining of the pelvis.

### Reducing the Risk of Developing Ovarian Cancer

Whereas many known risk factors for ovarian cancer cannot be modified (for example age, BRCA1/2 mutation status, family history, and previous breast cancer diagnosis), the percentage contribution of lactic acid-producing bacteria to the lower female genital tract microbiota can be modified. This is a major advantage of the present invention; not only have the inventors identified a new way to assess the risk of ovarian cancer, they have also identified new treatments for decreasing the risk of a subject developing ovarian cancer. A treatment which decreases the risk of developing ovarian cancer in the future or which prevents ovarian cancer may also be referred to as a “prophylactic treatment”.

### Compositions for Reducing the Risk of Ovarian Cancer

Hence, the present invention provides compositions for reducing the risk of developing ovarian cancer in a subject having been identified as being at increased risk of developing ovarian cancer.

Advantageously, the composition may comprise one or more species of lactic acid-producing bacteria, preferably one or more species of lactic acid-producing Lactobacilli, more preferably one or more species selected from the group consisting of L. crispatus, L. iners, L. gasseri, and L. jensenii. The composition may comprise L. crispatus, L. iners, L. gasseri, and L. jensenii. These compounds and compositions increase the percentage contribution of lactic acid-producing bacteria in the lower female genital tract, which bacteria produce lactic acid and thus lower pH.

The composition may comprise a systemic hormonal modulator. For example, the composition may comprise an oestrogen or an oral contraceptive (optionally comprising an oestrogen and a progestin, or a progestin alone). The inventors consider that such compounds support settling and/or growth of lactic acid-producing bacteria, which bacteria produce lactic acid and thus lower pH.

The composition may comprise combinations of the above. For example, the composition may comprise Gynoflor™, which comprises Lactobacillus acidophilus KS400 and estriol (E3).

The above compounds and compositions may be formulated as a vaginal suppository, cream, ointment, or wash.

A subject identified as being at increased risk of developing ovarian cancer, optionally a subject having been treated to decrease their risk of developing ovarian cancer, may be monitored for changes in their lower genital tract microbiome using the methods described herein.

A subject identified as being at increased risk of developing ovarian cancer, the risk of the subject developing ovarian cancer may also be referred to a doctor, preferably a gynaecologist. The subject may be referred for tests related to other risk factors, for example BRCA1/2 germline mutation testing. The subject may be referred for ovarian cancer screening, for example CA125 blood testing and/or a transvaginal ultrasound.

### Methods of Decreasing the Risk of Developing Ovarian Cancer

The present invention involves methods for decreasing the risk of a subject developing ovarian cancer, involving administering an effective amount of a composition to the subject. The compositions be any of the “compositions for reducing the risk of ovarian cancer” described above.

The method may involve identifying the subject as being at an increased risk for developing ovarian cancer. Alternatively, the subject may have already (previously) been identified as being at an increased risk for developing cancer.

### Computer Programs

Advantageously, computer programs may be used to implement the present invention.

Computer programs of the invention may process bacterial nucleic acid sequences derived from a sample obtained from the lower genital tract of a subject to determine which bacterial species they are derived from. Computer programs of the invention may calculate the percentage contribution of one or more of lactic acid-producing bacteria to the bacterial load of the sample. Computer programs of the invention may compare this percentage contribution to a percentage threshold value.

Computer programs of the invention may output a message stating whether the subject is at increased risk of developing or having ovarian cancer or being a carrier of a germline BRCA1 and/or BRCA2 mutation. Computer programs of the invention may output a message recommending a further course of action for the subject.

### EXAMPLES

The present invention is further described by reference to the following examples.

### Example 1-Methodology

16S rRNA gene sequencing (metataxonomics) was utilised in order to characterise the microbiota from cervico-vaginal samples. We calculated the sum percentage of lactobacilli species in each sample (i.e. L. crispatus, L. iners, L. gasseri and L. jensenii) and grouped the samples into those which were dominated by ≥50%) lactobacilli (“community-type L”) or <50% (referred to as “community-type O”).

Study design and participants

A study was conducted as part of a multi-centre study involving several recruitment sites in 5 European countries (i.e. the UK, Czech Republic, Italy, Norway and Germany). Volunteers were principally recruited from three different settings: (i) women with suspected ovarian cancer, benign gynaecological conditions and some women with a BRCA1 mutation were recruited in hospitals; (ii) healthy women from the general population were recruited as part of their routine cervical cancer screening programme; and (iii) the majority of BRCA1 mutation carriers and a proportion of heathy women were recruited via a dedicated research clinic. Participants were aged >18 years and prior to taking part, each prospective study volunteer was given a Participant Information Sheet as well as a Consent Form and the rationale for the study was explained. Additional resources, including an explanatory video and further online resources, were also made available. Women diagnosed with ovarian cancer or a non-malignant benign gynaecological condition were approached during outpatient hospital clinics, while women with a documented BRCA1 mutation and healthy controls were approached via outreach campaigns and public engagement. Prospective participants completed an epidemiological questionnaire, as well as a feedback form after their participation. The study itself is a sub-study of the FORECEE (4C) Programme, which has ethical approval from the UK Health Research Authority (REC 14/LO/1633).

Women were recruited to the FORECEE programme at several collection centres located across the UK and wider EU. Women with a suspected diagnosis of a malignant invasive epithelial ovarian cancer, recruited prior to an operation or receiving any chemo- or radio-therapy, were eligible as ovarian cancer cases. Controls were initially matched one-to-one with cases (ovarian cancer or BRCA mutation carriers) based on menopausal status, age (5 year age ranges where possible) and recruitment centre/country. However, due to an imbalance in recruitment of cases and controls at some centres, a number of cases were matched on age and menopausal status alone. The control subjects were collected from two different pools: 69 and 115 controls of the OC set and 14 and 97 controls of the BRCA set were collected from women attending hospitals due to benign conditions or were collected amongst the healthy general population, respectively. With regard to community-type L and O microbiota, women with benign gynaecological conditions or healthy volunteers did not differ according to a logistic regression model (for the OC set, p=0.49 and p=0.97 for age <50 and >50 years, respectively; for the BRCA set, and for the same age groups, p=0.99 and p=0.32, respectively).

The epidemiological survey was administered via the Qualtrics application on dedicated iPads. The survey comprised questions concerning health habits and relevant risk factors and also enquired as to historical health habits, as well as obtaining a thorough medical and obstetric history. Cervical samples were collected at appropriate clinical venues by trained staff. Cervical smears were carried out by a small group of research midwives or physicians with a view to establishing standard practice. Biological samples were given an anonymous Participant ID Number which was assigned to the person's name in a securely stored link file. Following sample taking, an email survey was sent to each participant, enabling them to feedback with respect to the recruitment process. For women who had been recruited as controls in the BRCA set (n=111), next generation sequencing (performed by Color Genomics) in white blood cell DNA was used in order to exclude a germline mutation in the BRCA1 and BRCA2 gene. Mutation testing was not undertaken for the OC set controls.

### Procedures

Cervical smear samples were taken at collaborating hospitals and recruitment centres using the ThinPrep system (Hologic, Inc). In brief, cervical cells were sampled from the cervix using a Rovers cervix brush which was rotated 5 times through 360 degrees before removing the brush from the vagina and immersing the brush head in a ThinPrep Vial containing Preserve-cyt fluid. The brush was then pushed against the bottom of the vial 10 times in order to facilitate release of the cells into the preserve-cyt solution before the brush was discarded. The sample vial was sealed and stored locally at room temperature before shipment to UCL at ambient temperature.

Regarding the average transit times and time to pre-processing, for the OC set, the Collection to Receipt time was between 0 and 71 days, with a mean value of 13.5 days, and the Receipt to Pre-process time, i.e. enriching the samples and storing them at −80° C., was between 0 and 434 days, with a mean value of 23.5 days. Utilising a t-test resulted in no significant differences in the variables between cases and controls: p=0.66 (Collection to Receipt) and p=0.12 (Receipt to Pre-process). For the BRCA set, Collection to Receipt times were lower, between 0 and 38 days (mean value=10.8 days). For Receipt to Pre-process times, samples ranged from 0 to 118 days (mean value=40.2 days). Collection to Receipt time differences between BRCA1 cases and controls were not significant (p=0.16), but for Receipt to Pre-process BRCA subjects had a mean value of 24.6 days, whilst controls had a value of 54.2 days (p<0.01). The majority of the BRCA samples were processed in a significantly shorter timeframe than the controls. Despite this discrepancy, if this covariate is taken individually as a predictor of community-type L or O under a logistic regression model (see methods section on biostatistics below), the results are not significant: p=0.86 and p=0.49 (Collection to Receipt, Age <50 years and >50 years, respectively); p=0.68 and p=0.71 (Receipt to Pre-process, Age <50 years and >50 years, respectively). Additionally, if the time variables for the BRCA set are combined to represent the total time period between a smear being taken (Date of Collection) and pre-process enrichment (Date of Child Creation), stored at −80° C. and checked again, there was no significant difference (under a t-test), between the BRCA1 and control groups. This is also verified under the classifier described in the statistics section: p=0.64 (Age <50 years) and p=0.592 (Age >50 years).

During wet laboratory processing, the cervical smear samples were poured into 50 mL Falcon tubes and left to sediment at room temperature for 2 hrs. 1 mL wide bore tips were subsequently utilised in order to transfer the cervical cell enriched sediment to a 2 mL cryo-vial before storage at −80° C.

For the next-generation sequencing protocol, total DNA extraction from cervical swabs was performed with the QlAsymphony DSP Virus/Pathogen kit according to the manufacturer's instructions (Qiagen, Hilden Germany). Sequencing and taxonomical classification of bacterial species in the cervical swab samples was performed by Eurofins Genomics Europe Sequencing GmbH (Constance, Germany). In short, the hyper-variable V1-V3 region of the bacterial SSU rDNA (16S rRNA genes) was amplified with primers (27F 5′- AGAGTTTGATCCTGGCTCAG and 534R 5′-ATTACCGCGGCTGCTGG) including Illumina adapters and dual index barcodes. These barcoded libraries were equimolarly pooled and sequenced on the HiSeq2500 with 300 bp paired-end reads (HiSeq Rapid SBS Kit v2, IIlumina). The profiling PCRs, NGS libraries, as well as the pooled libraries were quality checked via electropherograms and fluorometer concentration determinations. Additionally, positive and negative controls were included in each library preparation, including DNA extraction batches.

In order to process the 16S rRNA gene sequencing data, the demultiplexed sequencing reads were quality checked, trimmed and filtered (Sickle v1.33; https://github.com/najoshi/sickle; settings: -q 20, -I 246, -n yes) and adapters and primers removed (Cutadapt v1.10; https://bio.tools/cutadapt; settings: -minimum-length 246). Overlapping paired-end reads were merged for full-length V1-V3 16S amplicons (FLASh v1.2.11; settings: -min-overlap 10, -max-overlap 300, -max-mismatch-density 0.25), clustered (CD-HIT v4.6; settings: -c 0.99 and cluster filtered resulting in a minimum number of two members), and chimeric sequences removed (UCHIME v4.2.40). Operational taxonomic units (OTUs) were assigned with BLASTN+(v2.4.0; -evalue 1e-06) via a non-redundant 16S rRNA reference database from the Ribosomal Database Project (RDP, Release 11) and filtered for high quality (97% identity threshold, 95% alignment coverage, 376 query length threshold, 10% bitscore threshold). Taxonomic classification was based on the NCBI Taxonomy. To determine species diversity and evenness both the Shannon and the Simpson indices were calculated for each sample.

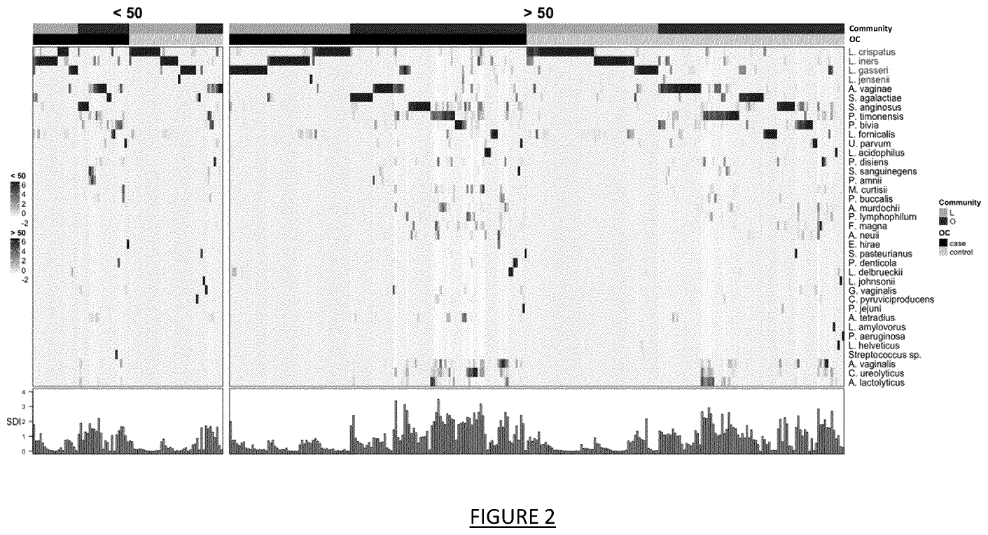

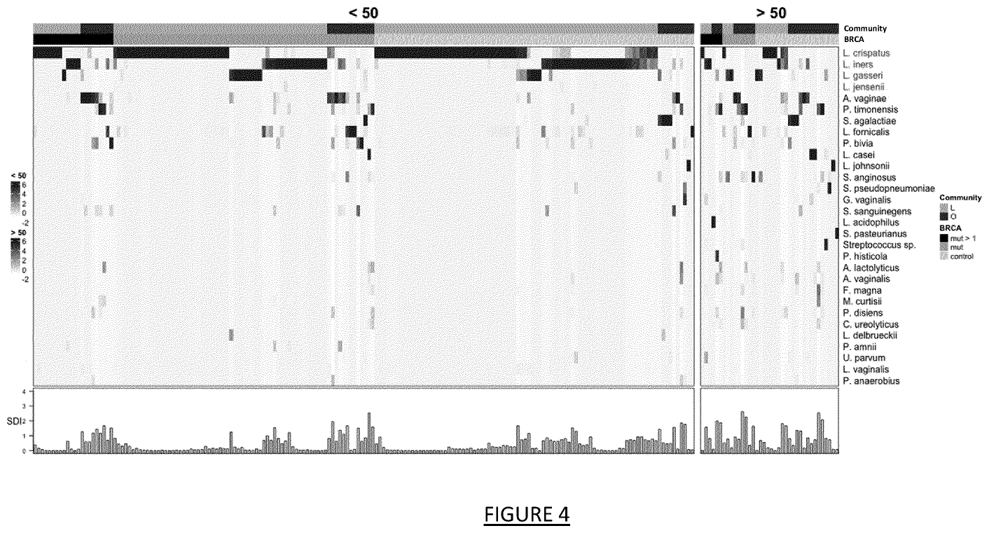

Regarding the abundance patterns presented in FIGS. 2 and 4, within each age group and each community-type L or O, they were clustered by a hierarchical clustering algorithm in R, hclust, via the Ward's method. The patterns are scaled column-wise. The species selected for the heatmaps correspond to Lactobacillus crispatus, iners, gasseri or jensenii (community-type L), in addition to those that ranked top on average abundance across all subjects in each set.

By observation of the 2D densities presented in FIG. 1, where the sum of percentages of species associated with community-type L is plotted against age, it is observed that a threshold of 50 years is able to distinguish the peaks associated with very low community-type L percentages.

### Statistical Analysis

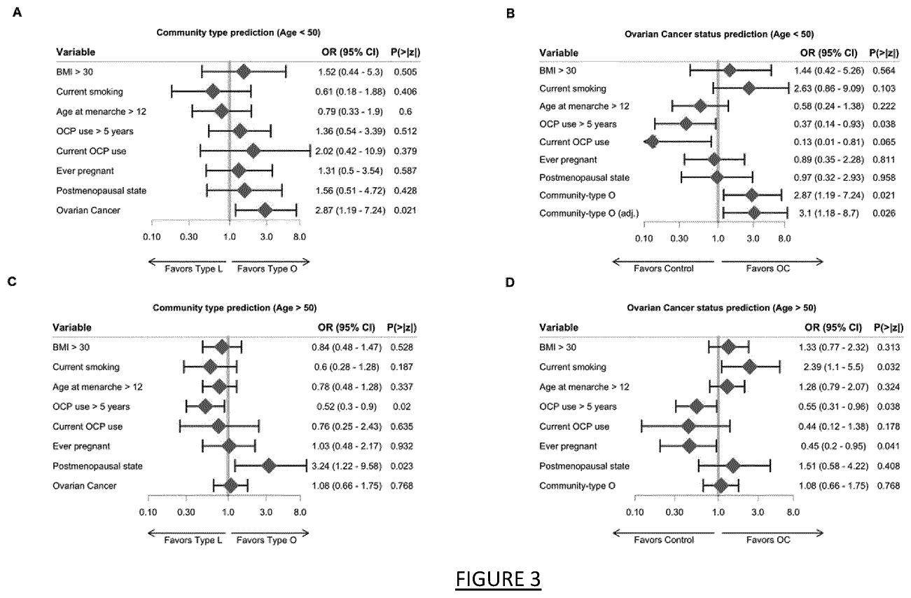

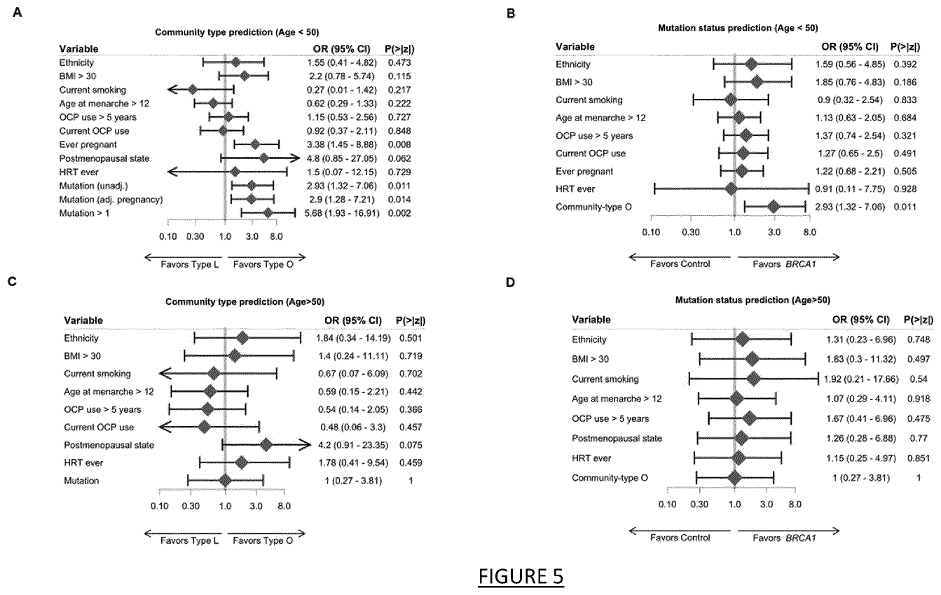

For the purposes of classification and identification of predictors, a logistic regression model was fitted independently to each covariate in the clinical data collected from each subject in the study (see FIGS. 3 and 5), as well as to both BRCA1/OC/Control status and community status-type L or O. The covariates leading to a statistically significant association with the outcome (p<0.05) were selected in order to adjust the prediction performance of both BRCA1/OC/Control status and community-type L or O status. This study used an additive model without interaction terms. The logistic regression model applied was implemented in the glm R package (https://stat.ethz.ch/R-manual/R-devel/library/stats/html/glm.html), with a binomial distribution. The quoted p-values , P(>|z|), correspond to a two-tailed test, where the respective z-values for each covariate taken as a predictor were the Wald statistic. Given that the number of samples N is larger than the number of predictors, even when adjusted (maximum of 3 covariates selected), the logistic regression model is stable.

### Example 2-Results of OC Set

This study recruited a total of 219 ovarian cancer cases and 219 age matched controls (“OC Set”) of which 176 and 184 provided sufficient DNA for 16S rRNA gene sequencing. The single most important factor which was associated with the proportion of type L communities in the overall population was age (p<10⁻⁴), which was even more pronounced than menopausal status (p=0.0002). Based on 2D densities plots presented in FIG. 1, it was decided to initially divide the cohort into < and >50 years of age. Sixty percent (165/275) of all volunteers >50 years of age exhibited a dominant type O community as the dominating feature whereas this was only the case for 41% (35/85) of the younger women.

In women under 50 years of age, the only factor which was significantly predictive of community status was the presence or absence of ovarian cancer, i.e. subjects having ovarian cancer exhibited a 2.9 fold increased risk of hosting a community-type O (FIG. 3A). Similarly, in addition to “Current OCP use” and “OCP use >5 years”, group community status was also verified to be significantly predictive of ovarian cancer status, which remained the most significant factor after adjusting for both of the additional covariates (OR 3.1; Cl 95% 1.2−8.7; p=0.026) (FIG. 3B). In women over 50 years of age there was a tendency towards a higher rate of type O communities in the cervico-vaginal samples, whereas conversely, women who had been on OCP for >5 years were not only more likely to have a type L community (FIG. 3C), a trend which was also observed for the 2^{nd} cohort studied (BRCA dataset, discussed further below), but also possessed a substantially lower risk of developing ovarian cancer (FIG. 3D). This observation indicates that long-term OCP use is associated with a delay in age-triggered community-type O predominance.

These data demonstrate that if a subject has community-type O cervico-vaginal microbiota (i.e. less than 50% of the bacterial load is contributed by L. crispatus, L. iners, L. gasseri and/or L. jensenii), the subject has a statically significantly increased risk of having ovarian cancer. L. crispatus, L. iners, L. gasseri and L. jensenii are four of the most prevalent lactic acid-producing bacterial species in the lower female genital tract, and the inventors consider that this effect would also be observed for other lactic acid-producing bacteria, particularly other Lactobacillus species.

It was also observed that the proportion of community-type L was almost identical, i.e. 42% and 39% respectively, within subjects having advanced (stage III/IV) and early (stage I/II) cancer. This observation indicates that the community-type of the lower genital tract microbiota is established before cancer develops. Hence, the inventors consider that the composition of the lower female genital tract microbiota can be used to assess a subject's risk of currently having ovarian cancer as well as developing ovarian cancer in the future.

### Example 3-Results of BRCA Set

BRCA1 germline mutation carriers who do not currently have ovarian cancer are a population of subjects who are known to be at a very high risk of developing ovarian cancer in the future. Hence, it was assessed whether a cervico-vaginal community-type O in BRCA1 germline mutation carriers (who could potentially benefit from a community-type L lactobacilli transplantation) is associated with an ovarian cancer predisposition.

To this end, this study analysed the microbiota of 111 controls and 109 age-matched BRCA1 mutation carriers, who have a ˜40-fold increased risk of developing ovarian cancer compared to mutation-free controls (FIG. 4). Both pregnancy and BRCA1 mutation status were associated with a higher risk for community-type O in <50 years women. After adjusting for pregnancy, BRCA1 mutation carriers had a 2.9 fold (95% Cl 1.3-7.2; p=0.014) higher risk for hosting an O community-type (FIG. 5A).

Within the ‘BRCA Set’, 34 BRCA1 mutation carriers had more than one first degree family member affected with cancer, a condition which further increases the future likelihood of developing ovarian cancer 1.8 fold. When comparing BRCA1 mutation carriers who had more than one first degree family members with a history of breast or ovarian cancer to women without a BRCA1 mutation, the risk for a community-type O microbiota increases 5.7-fold (95% Cl 1.9-16.9; p=0.0015; FIG. 5A). Among young women under 30 years of age, 6/22 (27.3%) and 0/23 (0%) with and without a BRCA1 mutation, respectively, exhibited community-type O dominance.

In the <50 years age group, community type was the only significant predictor of mutation status, with community-type O volunteers having a 2.9 fold (95% Cl 1.3 −7.1; p=0.011) increased risk of being a BRCA1 mutation carrier (FIG. 5B).

These data show that the percentage contribution of lactic acid-producing bacteria to the bacterial load of the lower female genital tract is a risk factor for being a BRCA1 germline mutation carrier. The inventors consider that the percentage contribution of lactic acid-producing bacteria to the bacterial load of the lower female genital tract is a risk factor for being a BRCA2 germline mutation carrier, since the mechanisms by which BRCA1 and BRCA2 mutations cause cancer are thought to be so closely related.

These data also demonstrate that subjects known to be at a very high risk of developing ovarian cancer (BRCA1 germline mutation carriers) are significantly more likely to have a community-type O lower genital tract microbiota. This supports the inventors' conclusion that a high percentage contribution of lactic acid-producing bacteria to the bacterial load of the lower female genital tract is a risk factor for developing ovarian cancer in the future (as well as already having ovarian cancer).

### Example 4-Varying the Age Threshold

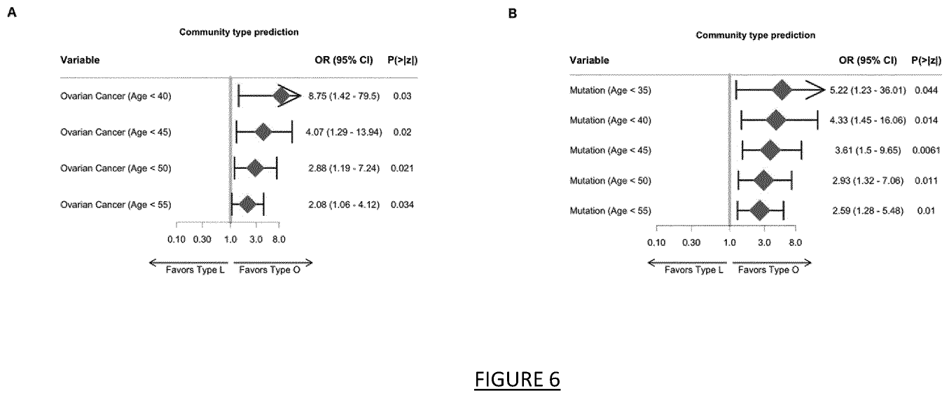

Finally, it was assessed whether the strong association between community-type O and cancer or BRCA1 mutation status depended on the age threshold (cut-off) that had been used. Hence, the analyses for each of the covariates collected for this study and performed with a threshold of 50 years (see FIGS. 3 and 5) was repeated for the OC and BRCA set with a threshold of 40, 45, 55 years and additionally for 35 years for the BRCA set which had sufficient cases and controls in this age group. For both the OC and BRCA sets, a statistically significant association was observed in all age groups tested. It was also found that the younger the age of women, the stronger the association between community-type O and ovarian cancer or BRCA1 mutation status (see FIG. 6).

These data confirm that the percentage contribution of lactic acid-producing bacteria to the bacterial load of the lower female genital tract is a statistically significant predictor of having ovarian cancer, developing ovarian cancer, or being a BRCA1/2 germline mutation carrier in women aged 55 or younger. The inventors consider that this risk factor would also be predictive in women up to the age of 60 years (sample availability limited testing of this age threshold).

These data also show that the percentage contribution of lactic acid-producing bacteria to the bacterial load of the lower female genital tract is a stronger/more meaningful predictor of having ovarian cancer, developing ovarian cancer, or being a BRCA1/2 germline mutation carrier in younger women, particularly in women younger than 45, and even more so in women younger than 40.

### Conclusions

In summary, these data demonstrate that (i) the presence of ovarian cancer itself or (ii) factors which are known to increase risk (i.e. age or the presence of a BRCA1 germline mutation) are associated with the lack of a protective lower female genital tract lactic acid-producing bacteria (particularly cervico-vaginal community-type L microbiota).

Without wishing to be bound by theory, the inventors hypothesise that an increased community-type O microbial species dominance results in reduced protection from ascending infections, which may be causally involved in ovarian cancer development. In addition, inflammation caused by community-type O bacteria and the release of bacterial toxins such as colibactin and cytolethal distending toxin may directly damage the genome by causing double-stranded breaks and thereby activate the DNA damage checkpoint pathway. This hypothesis is consistent with the fact that interventions such as tubal ligation and combined long term oral contraceptive pill (OCP) use are known to reduce ovarian cancer risk; tubal ligation may reduce ovarian cancer risk by limiting access of ascending bacteria to the Fallopian Tube and ovary and combined long term OCP use may reduce ovarian cancer risk by supporting the colonisation of the vagina with beneficial lactic acid-producing Lactobacillus species.

The inventors' findings may explain the inventors previous finding that BRCA1/2 germline mutation carriers have increased levels of progesterone in their luteal phase of the menstrual cycle (Widschwendter et al. (2013) Lancet Oncol; 14 (12): 1226-32.). The inventors hypothesise that these increased levels of progesterone reduce vaginal glycogen levels, which in turn reduces the levels of glucose and maltose (glycogen breakdown products), and thus the levels of lactic acid. A less acidic environment is thought to be less favourable for lactic acid-producing bacteria.

The application of vaginal suppositories using a combination of live lactobacilli and oestriol is effective in increasing the percentage of lactic acid-producing bacteria in the vaginal microbiota. Based on the findings presented herein, the inventors consider that such modification of the lower female genital tract microbiota would lower a woman's ovarian cancer risk.

1. A method of determining whether a subject is at an increased risk of developing or having ovarian cancer or being a carrier of a germline BRCA1 and/or BRCA2 mutation, the method comprising:
determining whether the percentage contribution of lactic acid-producing bacteria to the bacterial load of a sample obtained from the lower genital tract of the subject is more or less than a percentage threshold level,
wherein the subject is at increased risk of developing or having ovarian cancer or being a carrier of a germline BRCA1 and/or BRCA2 mutation if the percentage contribution of lactic acid-producing bacteria to the bacterial load of the sample is less than the percentage threshold level,
and wherein the subject is human and female.

2. A method of identifying a subject expected to benefit from modification of their lower genital tract microbiota, the method comprising:
determining whether the percentage contribution of lactic acid-producing bacteria to the bacterial load of a sample obtained from the lower genital tract of the subject is more or less than a percentage threshold level,
wherein the subject is expected to benefit from modification of their lower genital tract microbiota if the percentage contribution of lactic acid-producing bacteria to the bacterial load of the sample is less than the percentage threshold level,
and wherein the subject is human and female.

3. The method of claim 1 or 2, wherein the lactic acid-producing bacteria are:
(a) lactic acid-producing Lactobacillus species, or
(b) one or more species selected from the group consisting of L. crispatus, L. iners, L. gasseri, and L. jensenii.

4. The method of any of claims 1-3, wherein the percentage threshold level is:
(a) a value from 30 to 70%,
(b) a value from 40 to 60%,
(c) a value from 45 to 55%,
(d) a value from 48 to 52%,
(e) 50%, or
(f) the minimum percentage contribution of lactic acid-producing bacteria to the bacterial load of lower genital tract samples in a healthy control population.

5. The method of any one of claims 1-4, wherein the percentage contribution of lactic acid-producing bacteria to the bacterial load of the sample is determined using:
(a) targeted amplicon sequencing, optionally 16S rRNA amplicon sequencing,
or
(b) metagenomic sequencing, optionally shotgun metagenomic sequencing.

6. A method of determining whether a subject is at an increased risk of developing or having ovarian cancer or being a carrier of a germline BRCA1 and/or BRCA2 mutation, the method comprising:
determining the pH of a sample obtained from the lower genital tract of the subject,
wherein the subject is at increased risk of developing or having ovarian cancer or being a carrier of a germline BRCA1 and/or BRCA2 mutation if the pH is higher than a pH threshold level,
and wherein the subject is human and female.

7. A method of identifying a subject expected to benefit from modification of their lower genital tract microbiota, the method comprising:
determining the pH of a sample obtained from the lower genital tract of the subject,
wherein the subject is expected to benefit from modification of their lower genital tract microbiota if the pH is higher than a pH threshold level,
and wherein the subject is human and female.

8. The method of claim 6 or 7, wherein the pH threshold level is:
(a) a pH from 4.0 to 6.0,
(b) a pH from 4.5 to 5.5,
(c) a pH from 5.0 to 5.5, or
(d) a value 0.3-0.6 pH units higher than the average pH of samples obtained from a healthy control population.

9. The method of any of claims 1-8, wherein the method is a method of determining whether a subject is at an increased risk of developing or having ovarian cancer, and wherein the subject has previously been identified as a carrier of a germline BRCA1 and/or BRCA2 mutation.

10. The method of any of claims 1-9, wherein the subject is younger than 60, 55, 50, 45, or 40 years old.

11. The method of claim 10, wherein the subject is younger than 55 years old.

12. The method of claim 10, wherein the subject is younger than 40 years old.

13. The method of any of claims 1-12, wherein the subject is identified as being at a further increased risk of developing or having ovarian cancer or being a carrier of a germline BRCA1 and/or BRCA2 mutation if:
(a) the subject has two or more first degree family members having a history of ovarian or breast cancer;
(b) the subject has never used oral contraceptive pills (OCPs) or has used OCPs for a total of less than 5 years;
and/or
(c) the subject has used combined hormone replacement therapy (HRT) or has used combined HRT for a total of more than 5 years.

14. The method of claim 2 or 7, wherein the modification is an increase in the contribution of lactic acid-producing bacteria to the bacterial load of their lower genital tract microbiota,
optionally wherein the lactic acid-producing bacteria are:
(a) lactic acid-producing Lactobacillus species, or
(b) one or more species selected from the group consisting of L. crispatus, L. iners, L. gasseri, and L. jensenii,
further optionally wherein the increase is such that the percentage contribution of the lactic acid-producing bacteria to the bacterial load of their lower genital tract microbiota is:
(a) at least 30%,
(b) at least 40%,
(c) at least 45%,
(d) at least 48%,
(e) at least 50%, or
(f) at least the minimum percentage contribution of lactic acid-producing bacteria to the bacterial load of lower genital tract samples in a healthy control population.

15. The method of any of claim 2, 7, or 14, wherein the benefit is a decrease in the risk of the subject developing ovarian cancer.

16. A composition for use in a method of decreasing the risk of a subject developing ovarian cancer, wherein the subject has been identified as being at increased risk of developing ovarian cancer or as being expected to benefit from modification of their lower genital tract microbiota using the method according to any of claims 1-13,
wherein the composition comprises:
(a) one or more species of lactic acid-producing bacteria,
(b) an oestrogen, or
(c) an oral contraceptive,
or any combination thereof.

17. The composition for the use of claim 16, wherein the composition comprises one or more species of lactic acid-producing bacteria selected from the group consisting of:
(a) lactic acid-producing Lactobacillus species,
or
(b) L. crispatus, L. iners, L. gasseri, and L. jensenii.

18. The composition for the use of any of claims 16-17, wherein the method comprises administration of the composition to the lower genital tract.

19. A vaginal suppository comprising the composition for the use of any of claims 16-18.

20. A method of determining whether a subject is at an increased risk of developing or having ovarian cancer or being a carrier of a germline BRCA1 and/or BRCA2 mutation, the method comprising:
determining whether the percentage contribution of L. crispatus, L. iners, L. gasseri, and L. jensenii to the bacterial load of a sample obtained from the lower genital tract of the subject is more or less than 50%,
wherein the subject is at increased risk of developing or having ovarian cancer or being a carrier of a germline BRCA1 and/or BRCA2 mutation if the percentage contribution of L. crispatus, L. iners, L. gasseri, and L. jensenii to the bacterial load of the sample is less than 50%,
and wherein the subject is human and female and is 55 years old or younger.

21. A method of determining whether a subject is at an increased risk of developing or having ovarian cancer or being a carrier of a germline BRCA1 and/or BRCA2 mutation, the method comprising:
determining the pH of a sample obtained from the lower genital tract of the subject,
wherein the subject is at increased risk of developing or having ovarian cancer or being a carrier of a germline BRCA1 and/or BRCA2 mutation if the pH is higher than 5.0,
and wherein the subject is human and female and is 55 years old or younger.

22. A method for decreasing the risk of a subject developing ovarian cancer, the method comprising administering an effective amount of one or more lactic acid-producing bacteria to the subject,
wherein the subject is a female human and has been identified as being at an increased risk of developing ovarian cancer by:
determining whether the percentage contribution of lactic acid-producing bacteria to the bacterial load of a sample obtained from the lower genital tract of the subject is more or less than a percentage threshold level, and
identifying the subject as being at an increased risk of developing ovarian cancer if the percentage contribution of lactic acid-producing bacteria to the bacterial load of the sample is less than the percentage threshold level.

23. A method for decreasing the risk of a subject developing ovarian cancer, wherein the subject is at increased risk for developing ovarian cancer, the method comprising:
(a) obtaining or receiving a sample from the lower genital tract of a subject,
(b) determining whether the percentage contribution of lactic acid-producing bacteria to the bacterial load of a sample obtained from the lower genital tract of the subject is more or less than a percentage threshold level,
(c) identifying the subject as being at an increased risk of developing ovarian cancer if the percentage contribution of lactic acid-producing bacteria to the bacterial load of the sample is less than the percentage threshold level,
(d) administering an effective amount of one or more lactic acid-producing bacteria to the subject identified as being at an increased risk of ovarian cancer,
wherein the subject is human and female.

24. A method for decreasing the risk of a subject developing ovarian cancer, the method comprising administering an effective amount of one or more lactic acid-producing bacteria to the subject,
wherein the subject is a female human and has been identified as being at an increased risk of developing ovarian cancer by:
determining the pH of a sample obtained from the lower genital tract of subject, and
identifying the subject as being at an increased risk of developing ovarian cancer if the pH is higher than a pH threshold level.

25. A method for decreasing the risk of a subject developing ovarian cancer, wherein the subject is at increased risk for developing ovarian cancer, the method comprising:
(a) obtaining or receiving a sample from the lower genital tract of a subject,
(b) determining the pH of a sample obtained from the lower genital tract of the subject,
(c) identifying the subject as being at an increased risk of developing ovarian cancer if the pH is higher than a pH threshold level,
(d) administering an effective amount of one or more lactic acid-producing bacteria to the subject identified as being at an increased risk of ovarian cancer,
wherein the subject is human and female.

26. A computer program comprising instructions which, when the program is executed by a computer, cause the computer to carry out the method of any of claim 1-15 or 20-21.

27. A computer program comprising instructions which, when the program is executed by a computer, cause the computer to carry out the following steps:
processing bacterial nucleic acid sequences derived from a sample obtained from the lower genital tract of a subject to determine whether the percentage contribution of lactic acid-producing bacteria to the bacterial load of the sample is more or less than a percentage threshold level, and
identifying the subject as being at increased risk of developing or having ovarian cancer or being a carrier of a germline BRCA1 and/or BRCA2 mutation if the percentage contribution of lactic acid-producing bacteria to the bacterial load of the sample is less than the percentage threshold level,
wherein the subject is human and female.


SEQUENCE LISTING (2 sequences)

SEQ ID NO: 1 - DNA, 20 bp, Artificial Sequence
  Primer
agagtttgatcctggctcag

SEQ ID NO: 2 - DNA, 17 bp, Artificial Sequence
  Primer
attaccgcggctgctgg